(19) United States
(12) Patent Application Publication
(10) Pub. No.: US 2003/0231978 A1
(43) Pub. Date: Dec. 18, 2003

(54) Indoor air quality and antiseptic composition for use therein

(76) Inventors: Lanny U. Franklin, Atlanta, GA (US); Julio L. Pimentel, Buford, GA (US)

Correspondence Address: NEEDLE & ROSENBERG, P.C., SUITE 1000, 999 PEACHTREE STREET, ATLANTA, GA, 30309-3915, US

(21) Appl. No.: 10/370,085

(22) Filed: Feb. 19, 2003

Related U.S. Application Data
(60) Provisional application No. 60/358,089, filed on Feb. 19, 2002.

(51) Int. Cl. A61L 9/14; A61L 9/012; A61L 9/013; A61L 2/22

(52) U.S. Cl. 422/4; 422/28; 424/405; 424/43; 424/736; 424/742; 424/770

(57) ABSTRACT

Composition and methods for improving air quality, disinfecting surfaces, and prevention of a respiratory infection. The method can decrease pathogen and/or parasitic concentrations in closed rooms and surfaces by the application of an antiseptic composition. The composition can be a pressurized or foaming solution containing a single terpene, a terpene mixture, and/or a liposome:terpene(s) combination with or without surfactant. The composition can be a true solution of an effective amount of an effective terpene and a carrier such as water. The composition can be a suspension or emulsion of terpene, surfactant, and carrier. Application can be, for example, by spraying a confined space with a solution of the present invention.

### CROSS REFERENCE TO RELATED APPLICATIONS

This application claims the benefit of U.S. Provisional Application 60/358,089, filed Feb. 19, 2002, which is hereby incorporated by reference.

### BACKGROUND OF THE INVENTION

1. Field of the Invention

The invention relates to improvement of indoor air quality, disinfection of surfaces, and an antiseptic composition for use thereof.

2. Background

As civilization has progressed there has been a tendency to stay longer in closed and confined spaces. In fact, most people spend about 90% of their time indoors. This, in combination with central forced-air ventilation, has resulted in an increase in respiratory problems, especially allergies due to the presence of bacteria, fungi/mold, viruses, and parasites and their products in these confined places. In addition to respiratory problems, an increase in other infections and allergies can be attributed to indoor exposure to pathogens, allergens, and/or parasites. For these reasons, it is desirable to provide ways to improve indoor air quality and reduce indoor exposure to infective or allergenic agents.

The presence of microorganisms, toxins, and allergens is mainly due to poor ventilation, excess moisture, and improper cleaning and disinfection. Emissions from microbes into indoor air may include spores, volatile metabolites, and toxic secondary metabolites in particles. Inhaling mold spores can trigger allergic reactions such as asthma. Inhaling bacteria spores such as Anthrax can result in fatal infections.

Terms like “sick building syndrome” have become part of our vocabulary. The ever-increasing cases of sick building syndrome are due to the way houses and buildings are constructed to conserve energy. Cessation of interaction between indoor and outdoor air reduces the energy required to heat and cool a space. Use of central cooling and heating systems maintains livable temperatures and humidity levels within these confined spaces but also causes re-circulation of the same air and pollutants day after day, e.g., bacteria, viruses, mold, fungus, mildew, and gases.

In addition to airborne pathogens, allergens, and/or parasites, there are numerous pathogens, allergens, and parasites present in confined areas that collect in and on carpet, upholstery, and other surfaces, such as mites, bacteria, fungi, and spores.

Some biological contaminants trigger allergic reactions, including hypersensitivity pneumonitis, allergic rhinitis, and some types of asthma. Infectious illnesses, such as influenza, measles, and chicken pox are transmitted through the air. Molds and mildews release disease-causing toxins. Symptoms of health problems caused by biological pollutants include sneezing, watery eyes, coughing, shortness of breath, dizziness, lethargy, fever, and digestive problems. Children, elderly people, and people with breathing problems, allergies, and lung diseases are particularly susceptible to disease-causing biological agents in the indoor air.

One of the respiratory problems due to poor indoor air quality is sinusitis. Sinusitis is caused by bacteria (e.g., streptococci, staphylococci, pneumococci, Haemophilus influenza), viruses (e.g., rhinovirus, influenza virus, parainfluenza virus), and/or fungi (e.g., Aspergillus, Dematiaceae, Mucoraceae, Penicillium sp.). The incidence of sinusitis (or inflammation of the sinuses) appears to be increasing. According to a survey of consumers and primary care physicians, 42 percent of people surveyed reported having at least one sinus infection in the last 12 months, compared to 33 percent the previous year. Apr. 2, 2002 issue of Sinus News, http://www.sinusnews.com/Articles2/Allergies-Colds-Sinusitis.html. Health care experts estimate that 37 million Americans are affected by sinusitis every year. Americans spend millions of dollars each year for medications for their sinus symptoms.

Bacteria are the most common infectious agents in sinusitis. The bacteria most commonly implicated in sinusitis are the following: 1) Streptococcus pneumoniae (also called pneumococcal pneumonia or pneumococci), 2) Haemophilus influenzae, and 3) Moraxella catarrhalis. Less common bacterial culprits include other streptococcal strains (including Group A Streptococcus) and Staphylococcus aureus. Additionally, coagulase-negative staphylococci, alpha-hemolytic streptococci, and enteric bacilli can be found in chronic sinusitis. Patients with chronic sinusitis usually have several species of anaerobes and one or more aerobic pathogens.

Mycoplasmas can also be responsible for respiratory problems. Mycoplasmas are deemed bacteria but have a variety of differences relative to bacteria. One mycoplasma responsible for respiratory problems is Mycoplasma pneumoniae.

The typical process leading to bacterial sinusitis actually starts with a flu or cold virus. Viruses are directly implicated in about 10% of sinusitis cases.

Sometimes, fungal infections can cause sinusitis. Fungal sinusitis is known as eosinophilic fungal rhinosinusitis (EFRS) or eosinophilic mucinous rhinosinusitis (EMRS). Fungi are uncommon causes of sinusitis, but the incidence of these infections is increasing. Fungal infections are suspected in people with sinusitis who also have diabetes, leukemia, AIDS, or other conditions that impair the immune system. Fungal infections can also occur in patients with healthy immune systems, but they are far less common. Some people with fungal sinusitis have an allergic-type reaction to the fungi. Fungi involved in sinusitis are the following:

The fungus Aspergillus is the most common cause of all forms of fungal sinusitis.

Others include Curvularia, Bipolaris, Exserohilum, and Mucormycosis.

There have been a few reports of fungal sinusitis caused by Metarrhizium anisopliae, which is used in biological insect control.

The offending fungi generally originate from the classes Zygomycetes (Mucor spp.) and Ascomycetes (Aspergillus spp.). Three major types of fungus—Penicillum, Stachybotrys and Aspergillus—pose particular threats to human health and are the most predominant fungi found in air sampling. The Mayo Clinic Proceedings shows a report where 96% out of 210 patients with sinusitis had fungi. In some individuals, exposure to these fungi also can lead to asthma or to a lung disease resembling severe inflammatory asthma called allergic bronchopulmonary aspergillosis.

Mold exposure and health problems from these exposures have been prominent in the news and have spawned significant litigation. Mold grows in wet cellulose materials, including paper, insulation and paper products, cardboard, ceiling tiles, wood, wood products, dust, paints, wallpaper, insulation materials, drywall, carpet, fabric, and upholstery. Hot spots of mold growth include damp basements and closets, bathrooms, places where fresh food is stored, refrigerator drip trays, house plants, air conditioners, humidifiers, garbage pails, upholstered furniture, and bedding. When they grow uncontrolled, molds can gradually destroy the surfaces they are on by rendering them unusable. Removal of all visible molds, decontamination of surfaces, and reduction of moisture is the only way to combat a mold problem.

Molds can produce mycotoxin. Mycotoxins are lipid-soluble and are readily absorbed by the intestinal lining, airways, and skin. Species of mycotoxin-producing molds include Fusarium, Trichoderma, and Stachybotrys. The toxic effects from mold exposure are thought to be associated with exposure to toxins on the surface of the mold spores, not with growth of the mold in the body. Mycotoxins can cause a variety of symptoms and diseases, from short-term irritation of the airways to immunosuppression to some forms of cancer. Toxic molds are most dangerous when they are ingested. Some mold species can also infect the respiratory tract, causing chronic bronchitis and pneumonia.

Stachybotrys is the biggest health concern because of its high toxicity to humans and animals. Mycotoxins produced by Stachybotrys are extremely toxic, suppress the immune system, and may even be carcinogenic. Exposure may occur by skin contact, inhalation, or ingestion. Pulmonary hemorrhage (PH) is caused by toxins produced by an unusual fungus called Stachybotrys chartarum or similar fungi. Animals that eat large amounts of Stachybotrys-contaminated forage die rapidly from massive internal and external bleeding. Exposure to lower levels over time severely suppresses the immune system, resulting in opportunistic infections and other disease.

Diseases caused by Aspergillus are uncommon and rarely found in persons with normally functioning immune systems. However, Aspergillosis is the second most common fungal infection requiring medical treatment in the United States. Aspergillus may cause several different illnesses, including both infections and allergy. In some individuals, exposure to these fungi also can lead to asthma or to a lung disease resembling severe inflammatory asthma called allergic bronchopulmonary aspergillosis.

Another respiratory problem associated with indoor air quality is asthma. Asthma afflicts about 15 million Americans, including five million children. Since 1980, the biggest growth in asthma cases has been in children under five. The disease is a leading cause of childhood hospitalizations and school absenteeism, accounting for 100,000 child hospital visits a year, at a cost of almost $2 billion, and causing 10 million school days missed each year.

In addition to effects on human health, indoor air quality and surface exposure to pathogens and/or parasites is important in livestock confinement operations or other animal confinements, e.g., laboratory conditions. These close conditions are ideal for spreading diseases and/or parasites.

Prevention of these respiratory and non-respiratory problems have been handled in a number of ways, both chemical and non-chemical. Reducing or eliminating the infective agents and, thus, their products is responsible for improving these problems.

Since mold has an affinity for damp environments, one way to control indoor mold growth is to control moisture. By controlling the relative humidity level in a home, the growth of some sources of biologicals can be minimized. Standing water, water-damaged materials, or wet surfaces also serve as a breeding ground for molds, mildews, bacteria, and insects. House dust mites, the source of one of the most powerful biological allergens, grow in damp, warm environments.

A way of reducing exposure to biological contaminants includes installation and use of exhaust fans that are vented to the outdoors. Exhaust fans can eliminate much of the moisture that builds up from everyday activities.

Other sources of standing water, e.g., cool mist or ultrasonic humidifiers, evaporation trays in air conditioners, dehumidifiers, and refrigerators can be eliminated, refreshed, or cleaned frequently.

Thorough cleaning and drying water-damaged carpets and building materials or removal and replacement address another source of moisture.

Cleaning and disinfecting reduces various sources of infective agents. House dust mites and other allergy-causing agents can be reduced, although not eliminated, through regular cleaning. Various chemical agents and solutions can be used such as bleach, commercial cleaning solutions and the like.

Another way for reducing exposure to biological contaminants has been filtering the air.

UV lights are the newest tool to be used to improve indoor air quality. These lights will kill the source of numerous allergens or infective agents.

Of great concern is the emergence of common microbial strains that are now resistant to many chemicals or antibiotics. Although new powerful agents continue to designed for disinfection, they are expensive and are also prone to resistance eventually.

These previous compositions and methods have drawbacks. These include for example, resistance of microbes to agents, allergic reactions to chemical agents, and various side effects.

Terpenes are widespread in nature, mainly in plants as constituents of essential oils. Their building block is the hydrocarbon isoprene (C₅H₈)ₙ. Terpenes have been found to be effective and nontoxic dietary anti-tumor agents which act through a variety of mechanisms of action (Crowell, P. L. and M. N. Gould, 1994. Chemoprevention and therapy of cancer by d-limonene. Crit. Rev. Oncog. 5(1): 1-22; Crowell, P. L., S. Ayoubi and Y. D. Burke, 1996. Antitumorigenic effects of limonene and perillyl alcohol against pancreatic and breast cancer. Adv. Exp. Med. Biol. 401: 131-136). Terpenes, i.e., geraniol, tocotrienol, perillyl alcohol, b-ionone, and d-limonene, suppress hepatic HMG-COA reductase activity, a rate limiting step in cholesterol synthesis, and modestly lower cholesterol levels in animals (Elson, C. E. and S. G. Yu, 1994. The chemoprevention of cancer by mevalonate-derived constituents of fruits and vegetables. J. Nutr. 124: 607-614). D-limonene and geraniol reduced mammary tumors (Elegbede, J. A., C. E. Elson, A. Qureshi, M. A. Tanner and M. N. Gould, 1984. Inhibition of DMBA-induced mammary cancer by monoterpene d-limonene. Carcinogenesis 5(5): 661-664; Elegbede, J. A., C. E. Elson, A. Qureshi, M. A. Tanner and M. N. Gould, 1986. Regression of rat primary mammary tumors following dietary d-limonene. J. Natl. Cancer Inst. 76(2): 323-325; Karlson, J., A. K. Borg, R. Unelius, M. C. Shoshan, N. Wilking, U. Ringborg and S. Linder, 1996. Inhibition of tumor cell growth by monoterpenes in vitro: evidence of a Ras-independent mechanism of action. Anticancer Drugs 7(4): 422-429) and suppressed the growth of transplanted tumors (Yu, S. G., P. J. Anderson and C. E. Elson, 1995. The efficacy of B-ionone in the chemoprevention of rat mammary carcinogenesis. J. Agri. Food Chem. 43: 2144-2147).

Terpenes have also been found to inhibit the in vitro growth of bacteria and fungi (Chaumont J. P. and D. Leger, 1992. Campaign against allergic moulds in dwellings. Inhibitor properties of essential oil geranium “Bourbon”, citronellol, geraniol and citral. Ann Pharm Fr 50(3): 156-166; Moleyar, V. and P. Narasimham, 1992. Antibacterial activity of essential oil components. Int. J. Food Microbiol. 16(4): 337-342; and Pattnaik, S., V. R. Subramanyan, M. Bapaji and C. R. Kole, 1997. Antibacterial and antifungal activity of aromatic constituents of essential oils. Microbios. 89(358): 39-46) and some internal and external parasites (Hooser, S. B., V. R. Beasly and J. J. Everitt, 1986. Effects of an insecticidal dip containing d-limonene in the cat. J. Am. Vet. Med. Assoc. 189(8): 905-908). Geraniol was found to inhibit growth of Candida albicans and Saccharomyces cerevisiae strains by enhancing the rate of potassium leakage and disrupting membrane fluidity (Bard, M., M. R. Albert, N. Gupta, C. J. Guuynn and W. Stillwell, 1988. Geraniol interferes with membrane functions in strains of Candida and Saccharomyces. Lipids 23(6): 534-538). B-ionone has antifungal activity which was determined by inhibition of spore germination, and growth inhibition in agar (Mikhlin, E. D., V. P. Radina, A. A. Dmitrossky, L. P. Blinkova and L. G. Button, 1983. Antifungal and antimicrobial activity of some derivatives of beta-ionone and vitamin A. Prikl. Biokhim. Mikrobiol. 19: 795-803; Salt, S. D., S. Tuzun and J. Kuc, 1986. Effects of B-ionone and abscisic acid on the growth of tobacco and resistance to blue mold. Mimicry the effects of stem infection by Peronospora tabacina. Adam. Physiol. Molec. Plant Path. 28: 287-297). Teprenone (geranylgeranylacetone) has an antibacterial effect on H. pylori (Ishii, E.,1993. Antibacterial activity of teprenone, a non water-soluble antiulcer agent, against Helicobacter pylori. Int. J. Med. Microbiol. Virol. Parasitol. Infect. Dis. 280(1-2): 239-243). Rosanol, a commercial product with 1% rose oil, has been shown to inhibit the growth of several bacteria (Pseudomonas, Staphylococcus, E. coli, and H. pylori). Geraniol is the active component (75%) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae, E. A., M. J. Han, N. J. Kim and D. H. Kim, 1998. Anti-Helicobacter pylori activity of herbal medicines. Biol. Pharm. Bull. 21(9) 990-992). Extracts from cashew apple, anacardic acid, and (E)-2-hexenal have shown bactericidal effect against H. pylori.

Solutions of 11 different terpenes were effective in inhibiting the growth of pathogenic bacteria in in vitro tests; levels ranging between 100 ppm and 1000 ppm were effective. The terpenes were diluted in water with 1% polysorbate 20 (Kim, J., M. Marshall, and C. Wei, 1995. Antibacterial activity of some essential oil components against five food borne pathogens. J. Agric. Food Chem. 43: 2839-2845). Diterpenes, i.e., trichorabdal A (from R. Trichocarpa), has shown a very strong antibacterial effect against H. pylori (Kadota, et al., 1997).

There may be different modes of action of terpenes against microorganisms; they could (1) interfere with the phospholipid bilayer of the cell membrane, (2) impair a variety of enzyme systems (HMG-reductase), and (3) destroy or inactivate genetic material.

For the above reasons, and others, the present invention provides additional methods for controlling indoor air quality, disinfecting surfaces, and preventing respiratory infections that avoid the drawbacks of previous methods.

### SUMMARY OF THE INVENTION

In accordance with the purpose(s) of this invention, as embodied and broadly described herein, this invention relates to improvement of indoor air quality, disinfection of surfaces, and an antiseptic composition for use therein.

Disclosed is a method of decreasing pathogen and/or parasite concentration in a room or on a surface comprising applying a composition comprising an effective amount of at least one effective terpene.

A method of improving air quality in a confined space comprising applying a composition comprising an effective amount of at least one effective terpene is also disclosed.

Additionally disclosed is a method of improving air quality in a confined space by decreasing pathogen and/or parasitic concentration comprising applying a composition comprising an effective amount of an effective terpene.

The invention also provides a method of improving air quality by decreasing pathogen and parasite concentrations in closed rooms and surfaces comprising applying a pressurized or foaming solution comprising an effective amount of an effective terpene, an effective terpene mixture, a liposome- effective terpene(s) composition, or combination thereof.

The invention additionally provides a method for preventing a respiratory infection comprising decreasing pathogen and/or parasite concentration in a room or on a surface by applying a composition comprising an effective amount of at least one effective terpene.

A method for preventing a respiratory infection comprising improving air quality in a confined space containing a subject by applying a composition comprising an effective amount of at least one effective terpene is disclosed.

The present invention provides a composition for decreasing pathogen and/or parasite concentration, improving air quality, or preventing an infection. The composition can be a solution, especially a true solution. The composition can further comprise a carrier, e.g., water. The composition can further comprise a surfactant.

The composition may be a solution of terpene and water.

The composition of invention can comprise a mixture of different terpenes or a terpene-liposome (or other vehicle) combination.

The terpene of the composition can comprise, for example, citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, linalool, or mixtures thereof.

The composition is effective against various infective agents including bacteria, viruses, mycoplasmas, fungi, and/or parasites.

The methods are practiced using the compositions of the present invention.

The composition can be made by mixing an effective amount of an effective terpene and water. The mixing can be done at a solution-forming shear until formation of a true solution of the terpene and water, the solution-forming shear may be by high shear or high pressure blending or agitation.

A method is disclosed for improving air quality by decreasing fungal, bacterial and parasitical concentration in closed rooms and surfaces by the application of a pressurized solution containing a single terpene, a terpene mixture or a liposome-terpene(s) composition.

A method of improving air quality by decreasing fungal, bacterial and parasitic concentrations in closed rooms and surfaces by the application of a pressurized or foaming solution containing a terpene, a terpene mixture, or a liposome-terpene(s) composition is discussed herein.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF THE PREFERRED EMBODIMENTS

Before the present compounds, compositions, articles, devices, and/or methods are disclosed and described, it is to be understood that this invention is not limited to specific synthetic methods. It is also to be understood that the terminology used herein is for the purpose of describing particular aspects only and is not intended to be limiting.

In this specification and in the claims which follow, reference will be made to a number of terms which shall be defined to have the following meanings:

Definitions

It must be noted that, as used in the specification and the appended claims, the singular forms “a,” “an,” and “the” include plural referents unless the context clearly dictates otherwise. Thus, for example, reference to “an aerosol” includes mixtures of aerosols, reference to “a terpene” includes mixtures of two or more such terpenes, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by volume, of a particular element or component in a composition or article, denotes the volume relationship between the element or component and any other elements or components in the composition or article for which a part by volume is expressed. Thus, in a composition containing 2 parts by volume of component X and 5 parts by volume component Y, X and Y are present at a volume ratio of 2:5, and are present in such ratio regardless of whether additional components are contained in the composition.

A volume percent of a component, unless specifically stated to the contrary, is based on the total volume of the formulation or composition in which the component is included.

“Optional” or “optionally” means that the subsequently described event or circumstance may or may not occur, and that the description includes instances where said event or circumstance occurs and instances where it does not. For example, the phrase “optionally surfactant” means that the surfactant may or may not be added and that the description includes both with a surfactant and without a surfactant where there is a choice.

By the term “effective amount” of a compound or property as provided herein is meant such amount as is capable of performing the function of the compound or property- for which an effective amount is expressed, such as a sufficient amount of the compound to provide the desired function, i.e., antiseptic. As will be pointed out below, the exact amount required will vary from infective agent to infective agent, the concentration of the agent that is being targeted, the particular composition used, its mode of application, and the like. Thus, it is not possible to specify an exact “effective amount.” However, an appropriate effective amount may be determined by one of ordinary skill in the art using only routine experimentation.

By the term “effective terpene” is meant a terpene which is effective against the particular infective agent of interest.

As used throughout, by a “subject” is meant an individual. Thus, the “subject” can include, but is not limited to, domesticated animals (e.g., cats, dogs, etc.), livestock (e.g., cattle, horses, poultry, pigs, sheep, goats, etc.), and laboratory animals (e.g., mouse, rabbit, rat, guinea pig, etc.). In one aspect, the subject is a mammal, such as a primate or a human.

By the term “true solution” is meant a solution (essentially homogeneous mixture of a solute and a solvent) in contrast to an emulsion or suspension. A visual test for determination of a true solution is a clear resulting liquid. If the mixture remains cloudy, or otherwise not clear, it is assumed that the mixture formed is not a true solution but instead a mixture such as an emulsion or suspension.

By the term “confined” is meant any limited space; this term includes, for example, rooms or livestock confinements.

Poor indoor air quality is one of the major factors that induce allergies and produce respiratory infections in humans. Confined spaces also lead to other infections in subjects, such as humans or animals. The presence of microorganisms, toxins, and allergens is mainly due to poor ventilation, excess moisture, and improper cleaning and disinfection. Fungi, bacteria, and parasites (e.g., mites, lice) produce these allergens. There are several known procedures that can reduce the concentration of these allergens by reduction or elimination of their sources in the closed environment, but they are generally based on chemicals that are harmful to humans and animals.

The present invention avoids this problem by utilizing chemicals that are generally recognized as safe (GRAS) by the FDA and do not generate microbial resistance to the antiseptic. An aspect of this invention is that due to the mechanism of action, such as basic interference with cholesterol, terpenes do not generate microbial resistance. There are known antimicrobial products containing terpenes, basically in the form of essential oils, but we have found that not all components of an essential oil are biocidal.

The present invention has the capacity of reducing the incidences of respiratory infections by reduction and/or elimination of the pathogens and/or parasites responsible.

An aspect of the present invention is that by varying the concentration of terpenes different specificity and antiseptic effect can be achieved. Also, combinations of two or more terpenes in the same solution can generate a synergistic effect.

Another aspect of this invention is that the formulation can be tailored and obtain an antiseptic effect over a single type infective agent or alter the formulation and eliminate all types of infective agents.

We have observed that the terpenes used in this invention can be targeted to different microorganisms and parasites. We have demonstrated the effectiveness of the present invention against bacteria, molds, parasites, and other infective agents that are of importance to humans and animals. This invention can be modified in several ways by adding or deleting from the formulation various types of terpenes and surfactants.

The present invention includes methods of making the compositions and methods of using the compositions.

Composition(s)

The compositions of the present invention comprise isoprenoids. More specifically, the compositions of the present invention comprise terpenoids. Even more specifically, the compositions of the present invention comprise terpenes. Terpenes are widespread in nature, mainly in plants as constituents of essential oils. Terpenes are unsaturated aliphatic cyclic hydrocarbons. Their building block is the hydrocarbon isoprene (C₅H₈)ₙ. A terpene is any of various unsaturated hydrocarbons, such as C₁₀H₁₆, found in essential oils, oleoresins, and balsams of plants, such as conifers. Some terpenes are alcohols (e.g., menthol from peppermint oil), aldehydes (e.g., citronellal), or ketones.

Terpenes have been found to be effective and nontoxic dietary antitumor agents, which act through a variety of mechanisms of action. Crowell, P. L. and M. N. Gould, 1994. Chemoprevention and Therapy of Cancer by D-limonene, Crit. Rev. Oncog. 5(1): 1-22; Crowell, P. L., S. Ayoubi and Y. D. Burke, 1996, Antitumorigenic Effects of Limonene and Perillyl Alcohol Against Pancreatic and Breast Cancer, Adv. Exp. Med. Biol. 401: 131-136. Terpenes, i.e., geraniol, tocotrienol, perillyl alcohol, b-ionone and d-limonene, suppress hepatic HMG-COA reductase activity, a rate limiting step in cholesterol synthesis, and modestly lower cholesterol levels in animals. Elson C. E. and S. G. Yu, 1994, The Chemoprevention of Cancer by Mevalonate-Derived Constituents of Fruits and Vegetables, J. Nutr. 124: 607-614. D-limonene and geraniol reduced mammary tumors (Elgebede, J. A., C. E. Elson, A. Qureshi, M. A. Tanner and M. N. Gould, 1984, Inhibition of DMBA-Induced Mammary Cancer by Monoterpene D-limonene, Carcinogensis 5(5): 661-664; Elgebede, J. A., C. E. Elson, A. Qureshi, M. A. Tanner and M. N. Gould, 1986, Regression of Rat Primary Mammary Tumors Following Dietary D-limonene, J. Nat'l Cancer Institute 76(2): 323-325; Karlson, J., A. K. Borg, R. Unelius, M. C. Shoshan, N. Wilking, U. Ringborg and S. Linder, 1996, Inhibition of Tumor Cell Growth By Monoterpenes In Vitro: Evidence of a Ras-Independent Mechanism of Action, Anticancer Drugs 7(4): 422-429) and suppressed the growth of transplanted tumors (Yu, S. G., P. J. Anderson and C. E. Elson, 1995, The Efficacy of B-ionone in the Chemoprevention of Rat Mammary Carcinogensis, J. Angri. Food Chem. 43: 2144-2147).

Terpenes have also been found to inhibit the in vitro growth of bacteria and fungi (Chaumont J. P. and D. Leger, 1992, Campaign Against Allergic Moulds in Dwellings, Inhibitor Properties of Essential Oil Geranium “Bourbon, ” Citronellol, Geraniol and Citral, Ann. Pharm. Fr 50(3): 156-166), and some internal and external parasites (Hooser, S. B., V. R. Beasly and J. J. Everitt, 1986, Effects of an Insecticidal Dip Containing D-limonene in the Cat, J. Am. Vet. Med. Assoc. 189(8): 905-908). Geraniol was found to inhibit growth of Candida albicans and Saccharomyces cerevisiae strains by enhancing the rate of potassium leakage and disrupting membrane fluidity (Bard, M., M. R. Albert, N. Gupta, C. J. Guuynn and W. Stillwell, 1988, Geraniol Interferes with Membrane Functions in Strains of Candida and Saccharomyces, Lipids 23(6): 534-538). B-ionone has antifungal activity which was determined by inhibition of spore germination and growth inhibition in agar (Mikhlin E. D., V. P. Radina, A. A. Dmitrossky, L. P. Blinkova, and L. G. Button, 1983, Antifungal and Antimicrobial Activity of Some Derivatives of Beta-Ionone and Vitamin A, Prikl Biokhim Mikrobiol, 19: 795-803; Salt, S. D., S. Tuzun and J. Kuc, 1986, Effects of B-ionone and Abscisic Acid on the Growth of Tobacco and Resistance to Blue Mold, Mimicry the Effects of Stem Infection by Peronospora Tabacina, Adam Physiol. Molec. Plant Path 28:287-297). Teprenone (geranylgeranylacetone) has an antibacterial effect on H. pylori (Ishii, E., 1993, Antibacterial Activity of Terprenone, a Non Water-Soluble Antiulcer Agent, Against Helicobacter Pylori, Int. J. Med. Microbiol. Virol. Parasitol. Infect. Dis. 280(1-2): 239-243). Solutions of 11 different terpenes were effective in inhibiting the growth of pathogenic bacteria in in vitro tests; levels ranging between 100 ppm and 1000 ppm were effective. The terpenes were diluted in water with 1% polysorbate 20 (Kim, J., M. Marshall and C. Wei, 1995, Antibacterial Activity of Some Essential Oil Components Against Five Foodborne Pathogens, J. Agric. Food Chem. 43: 2839-2845). Diterpenes, i.e., trichorabdal A (from R. Trichocarpa), have shown a very strong antibacterial effect against H. pylori (Kadota, S., P. Basnet, E. Ishii, T. Tamura and T. Namba, 1997, Antibacterial Activity of Trichorabdal A from Rabdosia Trichocarpa Against Helicobacter Pylori, Zentralbl. Bakteriol 287(1): 63-67).

Rosanol, a commercial product with 1% rose oil, has been shown to inhibit the growth of several bacteria (Pseudomonas, Staphylococcus, E. coli, and H. pylori). Geraniol is the active component (75%) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae, E. A., M. J. Han, N. J. Kim, and D. H. Kim, 1998, Anti-Helicobacter Pylori Activity of Herbal Medicines, Biol., Pharm. Bull. 21(9) 990-992). Extracts from cashew apple, anacardic acid, and (E)-2-hexenal, have shown bactericidal effect against H. pylori.

There may be different modes of action of terpenes against microorganism; they could (1) interfere with the phospholipid bilayer of the cell membrane, (2) impair a variety of enzyme systems (HMG-reductase), and (3) destroy or inactivate genetic material. It is believed that due to the modes of action of terpenes being so basic, e.g., blocking of cholesterol, that infective agents will not be able to build a resistance to terpenes.

Terpenes, which are Generally Recognized as Safe (GRAS) have been found to inhibit the growth of cancerous cells, decrease tumor size, decrease cholesterol levels, and have a biocidal effect on microorganisms in vitro. Owawunmi, G. O., 1989, Evaluation of the Antimicrobial Activity of Citral, Letters in Applied Microbiology 9(3): 105-108, showed that growth media with more than 0.01% citral reduced the concentration of E. coli, and at 0.08% there was a bactericidal effect. Barranx, A. M. Barsacq, G. Dufau, and J. P. Lauilhe, 1998, Disinfectant or Antiseptic Composition Comprising at Least One Terpene Alcohol and at Lease One Bactericidal Acidic Surfactant, and Use of Such a Mixture, U.S. Pat. No. 5,673,468, teach a terpene formulation, based on pine oil, used as a disinfectant or antiseptic cleaner. Koga, J. T. Yamauchi, M. Shimura, Y. Ogasawara, N. Ogasawara and J. Suzuki, 1998, Antifungal Terpene Compounds and Process for Producing the Same, U.S. Pat. No. 5,849,956, teach that a terpene found in rice has antifungal activity. Iyer, L. M., J. R. Scott, and D. F. Whitfield, 1999, Antimicrobial Compositions, U.S. Pat. No. 5,939,050, teach an oral hygiene antimicrobial product with a combination of 2 or 3 terpenes that showed a synergistic effect. Several U.S. patents (U.S. Pat. Nos. 5,547,677, 5,549,901, 5,618,840, 5,629,021, 5,662,957, 5,700,679, 5,730,989) teach that certain types of oil-in-water emulsions have antimicrobial, adjuvant, and delivery properties.

Terpenes are widespread in nature. Their building block is the hydrocarbon isoprene (C₅H₈)ₙ. Examples of terpenes include citral, pinene, nerol, b-ionone, geraniol, carvacrol, eugenol, carvone, terpeniol, anethole, camphor, menthol, limonene, nerolidol, famesol, phytol, carotene (vitamin A₁), squalene, thymol, tocotrienol, perillyl alcohol, borneol, myrcene, simene, carene, terpenene, and linalool.

An effective terpene of the composition can comprise, for example, citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, linalool, or mixtures thereof. More specifically, the terpene can comprise citral, carvone, eugenol, b-ionone, or mixtures thereof.

The composition can comprise an effective amount of the terpene. By the term “effective amount” of a composition as provided herein is meant a sufficient amount of the composition to provide the desired result. An appropriate effective amount can be determined by one of ordinary skill in the art using only routine experimentation.

The composition can comprise between about 100 ppm and about 2000 ppm of the terpene, specifically about 100, 250, 500, or 1000 ppm.

A composition of the present invention comprises an effective amount of an effective terpene. An effective (i.e., antiseptic) amount of the effective terpene is the amount that produces a desired effect, e.g., decrease of infective agent concentration or prevention of an infection. This is the amount that will reach the necessary locations of the space at a concentration which will kill the infective agent. Less than a full kill may be effective for the desired end result as well. An amount that achieves a stable population or stasis of the infective agent may be sufficient to prevent disease. An effective (i.e., antiseptic) terpene is one which produces the desired effect, i.e., reduction or elimination of an infective agent or prevention of a respiratory infection against the particular infective agent(s) with the potential to infect or which have infected the subject(s).

The most effective terpenes can be the C₁₀H₁₆ terpenes. The more active terpenes for this invention can be the ones which contain oxygen. It is preferred for regulatory and safety reasons that at least food grade terpenes (as defined by the U.S. FDA) be used.

The composition can comprise a single terpene, more than one terpene, a liposome-terpene combination, or combinations thereof. Mixtures of terpenes can produce synergistic effects.

All classifications of natural or synthetic terpenes can be used in this invention, e.g., monoterpenes, sesquiterpenes, diterpenes, triterpenes, and tetraterpenes. Examples of terpenes that can be used in the present invention are citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, and linalool. The list of exempted terpenes found in EPA regulation 40 C.F.R. Part 152 is incorporated herein by reference in its entirety. The terpenes may also be known by their extract or essential oil names, such as lemongrass oil (contains citral).

Citral, for example citral 95, is an oxygenated C₁₀H₁₆ terpene, C₁₀H₁₆O CAS No. 5392-40-5 3,7-dimethyl-2,6-octadien-1-al.

Terpenes are readily commercially available or can be produced by various methods known in the art, such as solvent extraction or steam extraction/distillation. Natural or synthetic terpenes are expected to be effective in the invention. The method of acquiring the terpene is not critical to the operation of the invention.

The liposome-terpene(s) combination comprises encapsulation of the terpene, attachment of the terpene to a liposome, or is a mixture of liposome and terpene. Alternatively, vehicles other than liposomes may be used, such as microcapsules or microspheres. If the liposome or encapsulating vehicle serves as a time release device, the size and structure of the vehicle can be determined by one of skill in the art based on the desired release amounts and timing.

It is known to one of skill in the art how to produce a liposome or other encapsulating vehicle. For example, an oil-in-oil-in water composition of liposome-terpene may be used.

The composition can further comprise additional ingredients. For example, water (or alternatively, any bio-compatible or food-grade or pharmaceutically acceptable dilutant or carrier), a surfactant, preservative, or stabilizer.

The surfactant can be non-ionic, cationic, or anionic. Examples of surfactant include polysorbate (Tween®) 20, polysorbate 80, polysorbate 40, polysorbate 60, polyglyceryl ester, polyglyceryl monooleate, decaglyceryl monocaprylate, propylene glycol dicaprilate, triglycerol monostearate, Span® 20, Span® 40, Span® 60, Span® 80, or mixtures thereof.

A non-ionic surfactant can be used on all types of metal surfaces.

The composition can comprise 1 to 99% by volume terpenes and 0 to 99% by volume surfactant. More specifically the composition can comprise about 100 to about 2000 ppm terpenes and about 10% surfactant.

The composition may further comprise a foaming agent.

This composition can also further comprise other types of disinfectants, deodorizers, or carriers. This composition can be used in combination with carpet cleaners where a detergent is used for cleaning and the present invention as an antimicrobial and anti-parasitic.

The concentration of terpene in the composition is an antispetic amount. This amount can be from about an infective agent controlling level (e.g., about 100 ppm) to about a level with side effects or possibly even a level toxic to a subject's cells that may come into contact with the composition (e.g., about 2000 ppm generally causes irritation in humans, though the level may be cell or subject specific). Given time to dissipate, the upper concentration that can be used can possibly be greater than about 2000 ppm. This amount can vary depending on the terpene(s) used, the form of terpene (e.g., liposome-terpene), the infective agent targeted, and other parameters that would be apparent to one of skill in the art. One of skill in the art would readily be able to determine an antiseptic amount for a given application based on the general knowledge in the art and the procedures in the Examples given below.

Specific compositions can include e.g., bacteria and fungi—1000 ppm terpenes in standard 0.9% saline with 50% 1-carvone, 30% eugenol, 10% purified eucalyptus oil, and 10% Tween® 80; for mold—1000 ppm terpenes in water 100% citral or 95% citral and 5% Tween® 80; or for mycoplasma—125 ppm or 250 ppm in PBS 95% b-ionone and 5% Tween® 80.

Concentrations of terpene of 80, 90, 100, 110, 125, 130, 140, 150, 160, 175, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1250, 1375, 1425, 1500, 1600, 1750, or 2000 ppm canbe used as effective concentrations in the compositions and methods of the current invention.

Concentrations of any other ingredients or components can also be readily determined by one of skill in the art using methods known in the art and demonstrated below.

Terpenes have a relatively short life span of approximately 28 days once exposed to oxygen (e.g., air). Terpenes will decompose to CO₂ and water. This decomposition or break down of terpenes is an indication of the safety and environmental friendliness of the compositions and methods of the invention. The LD₅₀ in rats of citral is approximately 5 g/kg. This also is an indication of the relative safety of these compounds.

A stable suspension of citral can be formed up to about 2500 ppm. Citral can be made into a solution at up to about 1000 ppm.

Of the terpenes tested, citral has been found to form a solution at the highest concentration level. Citral will form a solution in water up to about 1000 ppm and will lyse human erythrocytes at approximately 1000 ppm.

At sufficiently high levels of terpene, a terpene acts as a solvent and will lyse cell walls.

A composition comprising a terpene, water, and a surfactant forms a suspension of the terpene in the water. Some terpenes may need a surfactant to form a relatively homogeneous mixture with water.

A composition comprising a “true” solution of a terpene is desirable in order to minimize additional components which may cause undesired effects. A method for making a true solution comprising a terpene is described below.

The composition(s) of the present invention are effective against most infective agents. Examples of infective agents include fungi, viruses, bacteria, and mycoplasmas. Also, infective agents can include parasites.

The terpenes, surfactants, or other components of the invention may be readily purchased or synthesized using techniques generally known to synthetic chemists. Methods for making specific and exemplary compositions of the present invention are described in detail in the Examples below.

The composition of the present invention can be sprayed on walls that shows mold growth in order to eliminate the production of allergens (spores) as well as reduce the number of spores in the air by the wetting action. The present invention can be sprayed on all types of surfaces in order to clean and disinfect. It can be applied as a water suspension or in combination with a foaming agent to help spread and stabilize the terpene or terpenes on the surfaces. It can be washed away after only a few minutes contact time or left to dry in place without damaging the target surface.

The amounts of the compositions described herein are large enough to produce the desired effect in the method by which delivery occurs. The amount should not be so large as to cause adverse side effects. The amount, schedule of use, and method of application can be varied.

Methods

The methods are practiced using the compositions of the present invention.

The invention includes a method of making the composition of the present invention. A method of making a terpene-containing composition that is effective for decreasing infective agent concentration in a confined space, improving air quality, or preventing a respiratory infection comprises adding an effective amount of an effective terpene to a carrier solvent.

The terpenes and carriers are discussed above. The concentration at which each component is present is also discussed above. For example, 1000 ppm of citral can be added to water to form a true solution. As another example, 2000 ppm of citral can be added to water with a surfactant to form a stable suspension.

The method can further comprise adding a surfactant to the terpene-containing composition. Concentrations and types of surfactants are discussed above. The method can further comprise adding additional ingredients discussed above such as foaming agents.

The method can further comprise mixing the terpene and carrier (e.g., water, saline, or buffer solution). The mixing is under sufficient shear until a “true” solution is formed. Mixing can be done via any of a number of high shear mixers or mixing methods. For example, adding terpene into a line containing water at a static mixer is expected to form a solution of the invention. With the more soluble terpenes, a true solution can be formed by agitating water and terpene by hand (e.g., in a flask). With lesser soluble terpenes, homogenizers, or blenders provide sufficient shear to form a true solution. With the least soluble terpenes, methods of adding very high shear are needed, or if enough shear cannot be created, can only be made into the desired mixture by addition of a surfactant.

Mixing the terpene and water with a solution-forming amount of shear instead of adding a surfactant will produce a true solution. A solution-forming amount of shear is that amount sufficient to create a true solution as evidenced by a final clear solution as opposed to a cloudy suspension or emulsion.

Citral is not normally miscible in water. Previously in the art, a surfactant has always been used to get such a terpene into solution in water. The present invention is able to form a solution of up to 1000 ppm in water by high shear mixing, and thus, overcome the necessity of a surfactant in all solutions.

Of the terpenes tested, citral has been found to form a solution at the highest concentration level in water.

In a large-scale production, the terpene can be added in line with the water and the high shear mixing can be accomplished by a static inline mixer.

Any type of high shear mixer will work. For example, a static mixer, hand mixer, blender, or homogenizer will work.

The invention includes a method of decreasing pathogen and/or parasite concentration in a room or on a surface comprising applying a composition comprising an effective amount of at least one effective terpene. The invention also includes a method of improving air quality in a confined space comprising applying a composition comprising an effective amount of at least one effective terpene.

The composition is the composition(s) described above.

The surface can be any surface compatible with the compositions of the present invention.

The application can be by any means or device known to one of skill in the art that is compatible with the compositions to be used and effective for reaching the areas to be treated.

The invention also provides a method of improving air quality by decreasing pathogen and parasite concentrations in closed rooms and surfaces comprising applying a pressurized or foaming solution comprising an effective amount of an effective terpene, an effective terpene mixture, a liposome- effective terpene(s) composition, or combination thereof.

The foaming solution can be formed by addition of a foaming agent or by addition of air or other gas sufficient to foam the composition to the desired degree.

The invention additionally provides a method for preventing a respiratory infection comprising decreasing pathogen and/or parasite concentration in a room or on a surface by applying a composition comprising an effective amount of at least one effective terpene.

A method for preventing a respiratory infection comprising improving air quality in a confined space containing a subject by applying a composition comprising an effective amount of at least one effective terpene is disclosed.

The present invention provides a composition for decreasing pathogen and/or parasite concentration, improving air quality, or preventing an infection. The composition can be a solution, especially a true solution. The composition can further comprise a carrier, e.g., water. The composition can further comprise a surfactant.

The composition may be a solution of terpene and water.

The composition can be made by mixing an effective amount of an effective terpene and water. The mixing can be done at a solution-forming shear until formation of a true solution of the terpene and water, the solution-forming shear may be by high shear or high pressure blending or agitation.

A method is disclosed for improving air quality by decreasing fungal, bacterial and parasitical concentration in closed rooms and surfaces by the application of a pressurized solution containing a single terpene, a terpene mixture or a liposome-terpene(s) composition.

A method of improving air quality by decreasing fungal, bacterial and parasitic concentrations in closed rooms and surfaces by the application of a pressurized or foaming solution containing a terpene, a terpene mixture, or a liposome-terpene(s) composition is discussed herein.

Infections in or on subjects are caused by a variety of organisms. For example, these organisms include bacteria, viruses, mycoplasmas, fungi, or parasites. The present invention is effective against any of these classifications of infective agents, in particular, bacteria, mycoplasmas, fungi, and parasites.

Examples of these infective agents are Staphylococcus aureus, Aspergillus fumigatus, Mycoplasma iowae, Sclerotinta homeocarpa, Rhizoctonia solani, Colletotrichum graminicola, Penicillum sp., and Mycoplasma pneumoniae.

The compositions and methods of the present invention are effective in preventing many, if not all, of these infections in a great variety of subjects, including humans and avians.

The invention includes a method of preventing a respiratory infection.

The composition of this invention can be applied by a variety of means. For example, the composition can be applied by spraying an aerosol, pressurized solution, or foaming solution into the confined space or onto a surface. Other means of application can be determined by one of skill in the art, for example, painting, pouring, or wiping the composition. The present invention can be sprayed on walls that shows mold growth in order to eliminate the production of allergens (spores) as well as reduce the number of spores in the air by the wetting action. The present invention can be sprayed on all types of surfaces in order to clean and disinfect. It can be applied as a water suspension or in combination with a foaming agent to help spread and stabilize the terpene or terpenes on the surfaces. It can be washed away after only a few minutes contact time or left to dry in place without damaging the target surface.

Applying one of the formulations of the present invention in spray form into a room or other area or onto a surface can reduce the amount of microorganism responsible for infections.

The life span/breakdown time of the terpenes, as indicated above, should be taken into account when formulating a schedule for use according to the present invention.

It will be apparent for those skilled in the art that the aforementioned objects and other advantages may be further achieved by the practice of the present invention.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by volume, temperature is in ° C. or is at ambient temperature, and pressure is at or near atmospheric. There are numerous variations and combinations of the compositions and conditions for making or using them, e.g., component concentrations, desired solvents, solvent mixtures, temperatures, pressures, and other ranges and conditions that can be used to optimize the results obtained from the described compositions and methods. Only reasonable and routine experimentation will be required to optimize these.

### Example 1

Preparation of the Terpene Mixture with Surfactant

The terpene, terpene mixture, or liposome-terpene(s) combination comprised a blend of generally recognized as safe (GRAS) terpenes with a GRAS surfactant. The volumetric ratio of terpenes was 1-99%, and the ratio of surfactant was 0-99% of the composition.

The terpenes, comprised of natural or synthetic terpenes, used were citral, b-ionone, eugenol, geraniol, carvone, terpeniol, or other terpenes with similar properties. The surfactant was polysorbate-80 (Tween® 80) or other suitable GRAS surfactant. The terpenes were added to water.

### Example 2

Preparation of a Terpene Solution without Surfactant

Alternatively, the solution can be prepared without a surfactant by placing the terpene, e.g., citral, in water and mixing under solution-forming shear conditions until the terpene is in solution.

The terpene-water solution was formulated without a surfactant. 100 ppm to 2000 ppm of natural or synthetic terpenes, such as citral, b-ionone, geraniol, carvone, terpeniol, carvacrol, anethole, or other terpenes with similar properties, were added to water and subjected to a high-shear blending action that forced the terpene(s) into a true solution. The terpene and water were blended in a household blender for 30 seconds. Alternatively, moderate agitation also prepared a solution of citral by shaking by hand for approximately 2-3 minutes.

The maximum level of terpene(s) that was solubilized varied with each terpene. Examples of these levels are as follows.

TABLE 1
Solution levels for various terpenes.

| Terpene | Level |
|---|---|
| Citral | 1000 ppm |
| Terpeniol | 500 ppm |
| b-ionone | 500 ppm |
| Geraniol | 500 ppm |
| Carvone | 500 ppm |

### Example 3

Preparation of Liposomes Containing Terpenes

Any standard method for the preparation of liposomes can be followed with the knowledge that the lipids used are all food-grade or pharmaceutical-grade.

A fixed amount of lipid(s), emulsifier, and terpene(s) were used to prepare an emulsion. The emulsion was obtained by using a Polytron® homogenizer with a stainless-steel flat bottom rotor specific for liposome and emulsion production.

The lipids were soybean oil, any commercial food-grade, or pharmaceutical oil; the emulsifier was egg yolk lecithin, plant sterols, or synthetic including polysorbate-80, polysorbate-20, polysorbate-40, polysorbate-60, polyglyceryl esters, polyglyceryl monooleate, decaglyceryl monocaprylate, propylene glycol dicaprilate, and triglycerol monostearate.

A solution containing 75-95 vol % lipids (oil) and 5-25% emulsifier made up the oil phase. The aqueous phase was a terpene(s) diluted in water at a rate of 0.5 vol % to 50%.

To form the emulsion, a volumetric ratio of oil to water varying from 10-15 parts lipid (oil phase) to 35-40 parts terpene(s) (aqueous phase) was mixed.

The suspension containing the lipid, emulsifier and terpene(s) was emulsified with the Polytron® homogenizer until a complete milky solution was obtained.

### Example 4

Preparation of Liposome

This Example illustrates the preparation of the terpene(s)-liposome combination by mixing 99 vol % of liposome and 1% of terpene mixture.

Several combinations of this formulation can be obtained by varying the amount of terpene and liposome from 1 vol % to 99%.

The liposomes are prepared as in Example 3 without the addition of terpenes in the formulation.

### Example 5

Potency of Solution

Terpenes will break down in the presence of oxygen.

Citral, for example, is an aldehyde and will decay (oxygenate) over a period of days. A 500 ppm solution will lose half its potency in 2-3 weeks.

### Example 6

In vitro Effectiveness of Terpenes Against Several Microorganisms

In vitro effectiveness of terpene compositions against various organisms was tested. The effectiveness of a terpene mixture solution comprising 10% by volume polysorbate-80, 10% b-ionone, 10% L-carvone, and 70% citral (lemon grass oil) against Escherichia coli, Salmonella typhimurium, Pasteurella mirabilis, Pseudomonas aeruginosa, Staphylococcus aureus, Candida albicans, and Aspergillus fumigatus was tested. The terpene mixture solution was prepared by adding terpenes to the surfactant. The terpene/surfactant was then added to water. The total volume was then stirred using a stir bar mixer.

Each organism, except A. fumigatus, was grown overnight at 35-37° C. in tryptose broth. A. fumigatus was grown for 48 hours. Each organism was adjusted to approximately 10⁵ organisms/ml with sterile saline. For the broth dilution test, terpene mixture was diluted in sterile tryptose broth to give the following dilutions: 1:500, 1:1000, 1:2000, 1:4000, 1:8000, 1:16000, 1:32000, 1:64000, and 1:128000. Each dilution was added to sterile tubes in 5 ml amounts. Three replicates of each series of dilutions were used for each test organism. One half ml of the test organism was added to each series and incubated at 35-37° C. for 18-24 hours. After incubation the tubes were observed for growth and plated onto blood agar. The tubes were incubated an additional 24 hours and observed again. The A. fumigatus test series was incubated for 72 hours. The minimum inhibitory concentration (MIC) for each test organism was determined as the highest dilution that completely inhibited the organism.

TABLE 2
Results of the inhibitory activity of different dilutions of terpene composition.

| | Visual Assessment | | | Growth After Subculture | | | |
|---|---|---|---|---|---|---|---|
| | of Growth* | | | to Agar Plates* | | | Mean Inhibitory |
| Organism | 1 | 2 | 3 | 1 | 2 | 3 | Dilution |
| S. typhimurium | 500 | 500 | 500 | 500 | 500 | 500 | 500 |
| E. coli | 1000 | 1000 | 1000 | 1000 | 1000 | 1000 | 1000 |
| P. mirabilis | 1000 | 1000 | 1000 | 1000 | 1000 | 1000 | 1000 |
| P. aureginosa | NI** | NI | NI | NI | NI | NI | NI |
| S. aureus | 1000 | 1000 | 1000 | 1000 | 1000 | 1000 | 1000 |
| C. albicans | 1000 | 1000 | 1000 | 1000 | 1000 | 1000 | 1000 |
| A. fumigatus | 8000 | 16000 | 16000 | 8000 | 16000 | 16000 | 13300 |

*The results of the triplicate test with each organism as the reciprocal of the dilution that showed inhibition/killing.
**NI = not inhibited.

### Example 7

Effects of Terpene on Growth of Mycoplasma iowae

Effects of neat citral on growth of Mycoplasma iowae was studied. M iowae is a known avian respiratory disease agent.

Three concentrations (500 ppm, 250 ppm, and 125 ppm) of citral in sterile DI water were prepared.

Mycoplasma iowae were incubated at 37° C. in R₂ (Chen, T. A., J. M. Wells, and C. H. Liao. 1982. Cultivation in vitro: spiroplasmas, plant mycoplasmas, and other fastidious, walled prokaryotes. pp. 417-446. in Phytopathogenic prokaryotes, V. 2, M. S. Mount and G. H. Lacy (ed.), Academic Press, New York) broth.

One to 2-day old cultures were observed under a dark-field microscope to ensure cells were in filamentous form before treatment. Cell suspensions were vortexed to ensure they were evenly mixed before, and an aliquot of 0.5 mL was dispensed into a sterile tube.

One half of 1 mL of each terpene solution was added into each cell suspension tube. Thus, the final concentrations of citral were 250 ppm, 125 ppm, and 62.5 ppm, respectively. The cell suspension that was added with 0.5 mL of sterile water was used as a control.

The treated cell suspension was incubated for 24 hrs before the color changing units (CCUs) were determined by a 10-fold serial dilution in fresh R₂. All treatments were duplicated. The CCUs were determined to 10⁻⁸ for terpene concentrations of 250 ppm and 125 ppm, and to 10⁻⁹ for a terpene concentration of 62.5 ppm and sterile water.

All culture tubes were incubated for 15 days before final readings were taken.

TABLE 3
Results of citral in vitro against Mycoplasma iowae.

| | Treatment | | | |
|---|---|---|---|---|
| | Water-treated | 62.5 ppm | 125 ppm | 250 ppm |
| Organism | (CCUs) | | | |
| M. iowae | 10⁹ | 10⁸ | 10⁸ | 10⁷ |

A comparison was made of the effect of 24-hr and 48-hr treatment times. The CCUs were determined by taking treated cell suspension from the same treated tube 24 hrs or 48 hrs after treatment.

TABLE 4
24 and 48 hour treatment comparisons.

| | Treatment (ppm) | | | | | | | |
|---|---|---|---|---|---|---|---|---|
| | Water- | Water- | | | | | | |
| | treated | treated | 62.5 | 62.5 | 125 | 125 | 250 | 250 |
| | 24 hr | 48 hr | 24 hr | 48 hr | 24 hr | 48 hr | 24 hr | 48 hr |
| Organism | (CCUs) | | | | | | | |
| M. iowae | 10⁷ | 10⁶ | 10⁶ | 10⁶ | 10⁷ | 10⁶ | 10⁵ | 10⁴ |

The results indicate that citral may be able to serve as a chemical for control of avian respiratory diseases when used at higher than 250 ppm and treated for a sufficient length of time.

### Example 8

In vitro Effectiveness of Different Terpene Formulations Against Escherichia coli, Salmonella typhimurium, Pasteurella mirabilis, Pseudomonas aeruginosa, Staphylococcus aureus, Candida albicans, and Aspergillus fumigatus

This example shows the amount and types of terpenes from six different terpene formulations (Table 5) used for antimicrobial testing.

In the microbiological study, seven microorganisms including Escherichia coli, Salmonella typhimurium, Pasteurella mirabilis, Pseudomonas aeruginosa, Staphylococcus aureus, Candida albicans, and Aspergillus fumigatus were utilized. These microorganisms were selected in view that they are commonly present in infections and contaminate animal products utilized for human consumption. Each organism, except A. fumigatus, was grown overnight at 35-37° C. in tryptone broth. A. fumigatus was grown for 48 hours. Each organism was adjusted to approximately 10⁵ organisms/ml with sterile saline.

Each terpene formulation was diluted to 1:500, 1:1000, 1:2000, 1:4000, 1:8000, and 1:16000 in broth and/or saline.

Each terpene formulation dilution was added to sterile tubes in 5 ml amounts, and 5 ml of the test organism was added to each series and incubated for 1 hour. There were three replicates of each series of dilutions for each test organism.

After incubation, 0.5 ml of each tube was plated onto blood agar and incubated 18-24 hours at 35-37° C. The A. fumigatus test series was incubated for 72 hours at 25° C.

The minimum inhibitory concentration (MIC) for each test organism was determined as the highest dilution that completely inhibits the organism growth. The microbiological results are presented in Table 6.

TABLE 5
Terpene formulation used for antimicrobial testing.

Formulas (vol %)

| Terpene/Ingredient | A | B | C | D | E | F |
|---|---|---|---|---|---|---|
| Citral | | 15 | | | 20 | 70 |
| Carvone | | | 55 | 55 | 35 | 10 |
| Eugenol | | | 35 | | 40 | 10 |
| b-ionone | 30 | 80 | 10 | 40 | | |
| Liposome | 70 | | | | | |
| Tween ® 80 | | 5 | 5 | 5 | 5 | 10 |

TABLE 6
Effect of terpene formulations on microorganism growth.
DILUTION AT WHICH MICROORGANISM GROWTH
WAS INHIBITED

| | Formula | | | | | |
|---|---|---|---|---|---|---|
| Organism | A | B | C | D | E | F |
| E. coli | NI | NI | NI | NI | 2000 | 1000 |
| P. aeruginosa | NI | NI | NI | NI | 2000 | NI |
| P. mirabilis | NI | NI | NI | NI | 1000 | 1000 |
| S. typhimurium | NI | NI | NI | NI | 2000 | 500 |
| S. aureus | NI | 4000 | 1000 | 4000 | 2000 | 1000 |
| C. albicans | NI | 1000 | 2000 | 2000 | 2000 | 1000 |
| A. fumigatus | NI | NI | NI | NI | 500 | 13300 |

The results are expressed as the reciprocal of the dilution that showed biocidal effect.
NI = not inhibited

### Example 9

In vitro Effectiveness of Terpenes Against Fungal Microorganisms: Scierotinta homeocarpa, Rhizoctonia solani, and Colletotrichum graminicola

Two terpene formulations were tested against Sclerotinta homeocarpa, Rhizoctonia solani, and Colletotrichum graminicola. Formula A contained 40 vol % eugenol, 35% 1-carvone, 20% citral, and 5% Tween® 80. Formula B contained 70 vol % citral, 10% b-ionone, 10% 1-carvone, and 10% Tween® 80.

Potato dextrose agar media was amended with each terpene formulation to make a 5000 ppm final concentration of each.

For each pathogen, a 5 mm diameter agar plug containing fungal mycelia was transferred to each of 5 plates for both terpene formulations and a control. All plates were parafilmed and incubated at 25° C. The diameter of fungal colony growth was measured (mm) and recorded. When the control plates were fill, measurements were stopped. Colony area was calculated using π r², where r is the radius of the colony.

TABLE 7
Effect of terpenes on fungal growth (area = mm²).

| | S. homeocarpa | | R. solani | | C. graminicola | |
|---|---|---|---|---|---|---|
| Treatment | Day 1 | Day 2 | Day 1 | Day 2 | Day 2 | Day 7 |
| Formula A | 0 | 0 | 0 | 0 | 0 | 0 |
| Formula B | 0 | 0 | 0 | 0 | 0 | 0 |
| Control | 209.0 | 2023.2 | 162.3 | 1976.6 | 136.7 | 2023.2 |

### Example 10

In vitro Effectiveness of Single or Combination of Terpenes Against E. coli

The objective of this example was to determine a terpene mixture that could have an optimal biocidal effect.

E. coli strain AW574 was grown in tryptone broth to an exponential growth phase (O.D. between 0.4 and 1.0 at 590 nm). One tenth of this growth was inoculated to 10 ml of tryptone broth followed by the addition of individual terpenes or as indicated on Table 8; then incubated for 24 hours at 35-37° C., and the O.D. determined in each tube. The concentration of terpenes was 1 or 2 μMol. Each treatment was repeated in triplicate. The results are expressed as percentage bacterial growth as compared to the control treatment.

It is observed that the combination of terpenes gives better biocidal effect than single terpenes, with geraniol and carvone appearing to be better than b-ionone.

TABLE 8
Effect of single terpene or their combination against E. coli growth.

| μMol terpenes | | | % |
|---|---|---|---|
| b-ionone | Carvone | Geraniol | growth |
| 0 | 0 | 0 | 100.00 |
| 2 | 0 | 0 | 84.00 |
| 0 | 2 | 0 | 63.00 |
| 0 | 0 | 2 | 54.00 |
| 1 | 1 | 1 | 41.00 |
| 1 | 2 | 1 | 31.10 |
| 1 | 1 | 2 | 14.80 |
| 1 | 2 | 2 | 15.90 |
| 2 | 1 | 1 | 48.60 |
| 2 | 2 | 1 | 44.30 |
| 2 | 1 | 2 | 30.20 |
| 2 | 2 | 2 | 1.50 |

### Example 11

In-vitro Larvacidal Effect of Terpenes

Third stage (L3) Dirofilaria immitis larvae were suspended at a concentration of 85 larvae/ml. Larvae were deposited on plates with wells containing b-ionone at a concentration of 5 μM, 50 μM, and 750 μM, Tween® 80, and control media.

Plates were incubated at 37° C., 95% relative humidity (RH), and 5% CO₂ for 6 days.

After incubation all wells were evaluated for appearance, motility, and molting to stage four (L4) at 24, 48, 72 hours, and 6 days.

B-ionone at 750 μM had a very detrimental effect on the survival, motility, and molting of D. immitis, as all larvae were dead or moribund within 24 hours.

At 50 μM it showed a decreased survivability, motility, and molting throughout the 6 day duration of the assay. Most larvae were dead by 6 days and none molted.

At a b-ionone concentration of 5 μM or Tween® 80, there was no effect on the larvae since results were similar to the control group.

### Example 12

Summary of Mold Studies

TABLE 9
Formulas tested.

| Terpene | | | | | | | |
|---|---|---|---|---|---|---|---|
| (vol %) | A | FW | B | C | D | E | F |
| Citral | 20 | 70 | 10 | 30 | 60 | 100 | 95 |
| l-Carvone | 35 | 10 | 50 | 30 | 30 | — | — |
| Eugenol | 40 | — | 30 | 30 | — | — | — |
| b-ionone | — | 10 | — | — | — | — | — |
| Tween ® 80 | 5 | 10 | 10 | 10 | 10 | — | 5 |

Study 1:

1. Mold spores, Penicillum sp., were mixed with 1000 ppm of terpene formulation as indicated in Table 9 and added to a Potato-Dextrose agar plate.

2. After 48 h incubation, the plates showed the following results:

F<E<C<FW<D<A<B<Control.

3. After 72 hours, the plates showed the following results:

F<E<C<FW<D<A<B<Control.

Formulas F and E performed better than the others.

Study 2:

1. Mold spores, Penicillum sp., were mixed with 1000 ppm of each terpene formulation, incubated for 1 hour, and then added to Potato-Dextrose agar plates.

2. After 48 h incubation, the plates showed the following results:

F<FW<E<D<C<B<A<Control.

Formulas F and E performed better than the others.

Study 3:

1. Mold spores, Penicillum sp., were mixed with 1000 ppm of each terpene formulation, incubated for 24 hours, and then added to Potato-Dextrose agar plates.

2. After 48 h incubation, the plates showed the following results:

F<E<D<FW<C<A<B<Control

Formulas F and E performed better than the others.

Tests were repeated several times with the same results. Formulas E and F performed better that the others.

### Example 13

Biofilm Formation and Testing (Destruction of Biofilm)

Procedure:

In 96-well polystyrene plates or PVC plates

1. Add 100 ml of bacterial culture in nutrient broth, culture has to be made fresh by adding 1-2 ml of 1×10⁶ cfu in 50-100 ml broth and incubating overnight (14-18 h) at 37° C.

2. Incubate overnight at 35-37° C. This will develop a Biofilm.

3. Wash 4 times with water.

4. Add 100 ml of 1:1000 terpene solution.

5. Let incubate for 1 hour or more depending on test protocol.

6. Add 25 μl of 1% crystal violet. This is done to quantify the biofilm formation. Dye will coat bacteria attached to wells.

7. Incubate for 15 minutes.

8. Wash wells four times with water and blot dry.

9. Add 200 μl 95% ethanol, mix.

10. In a new plate, transfer 150 μl solution to clean wells.

11. Read at 590 nm.

12. Results are expressed as the difference between O.D. of control and the treated samples.

Study 1:

Four terpene formulations with two type of surfactants, a total of eight formulas (A, B, C and D with 10% Tween® 80, H, J, K and L have 10% Span® 20) were 5 prepared. Formulas A-D are those used in Example 12 with 10% Tween® 80. H-L are Formulas A-D from Example 12 with 10% Span® 20.

TABLE 10
Formulas tested vs. control for reduction in biofilm achieved.

| Formula | O.D. test | O.D. control | % reduction |
|---|---|---|---|
| A | 0.098 | 0.210 | 53 |
| B | 0.187 | 0.220 | 15 |
| C | 0.220 | 0.229 | 4 |
| D | 0.295 | 0.230 | 0 |
| H | 0.223 | 0.230 | 3 |
| J | 0.273 | 0.194 | 0 |
| K | 0.233 | 0.194 | 0 |
| L | 0.153 | 0.194 | 0 |

Study 2:

Destruction of Biofilm by terpenes

Five formulas with their results. The formulas correspond to those used in Example 12.

TABLE 11
Formulas tested vs. control for reduction in biofilm achieved.

| Formula | O.D. test | O.D. control | % reduction |
|---|---|---|---|
| A | 0.299 | 0.459 | 35 |
| FW | 0.437 | 0.459 | 5 |
| B | 0.284 | 0.459 | 38 |
| C | 0.264 | 0.459 | 42 |
| D | 0.247 | 0.459 | 46 |

### Example 14

Biofilm Formation and Testing (Prevention of Biofilm Formation)

Procedure:

In 96-well polystyrene plates or PVC plates

1. Add 50 ml of bacterial culture in nutrient broth, culture has to be made fresh by adding 1-2 ml of 1×10⁶ cfu in 50-100 ml broth and incubating overnight (14-18 h) at 37° C.

2. Add 100 ml of 1:1000 terpene solution.

3. Incubate overnight at 35-37° C. This will develop a Biofilm.

4. Wash 4 times with water.

5. Add 25 μl of 1% crystal violet. This is done to quantify the biofilm formation. Dye will coat bacteria attached to wells.

6. Incubate for 15 minutes.

7. Wash wells four times with water and blot dry.

8. Add 200 μl 95% ethanol, mix.

9. In a new plate, transfer 150 μl solution to clean wells.

10. Read at 590 nm.

11. Results are expressed as the difference between O.D. of control as compared to treated samples.

### Example 15

Determination of Citral in Water Samples

Reagents: Schiff reagent is diluted 1:10 with distilled water.

1. In test tubes, add 1 ml of solution to be tested.

2. Add 0.1 ml of 1:10 Schiff reagent.

3. Incubate at room temperature for 10 minutes.

4. Reaction will turn from pink to blue, pink color is 0 ppm citral, reaction starts to turn blue above 100 ppm.

### Example 16

In vitro Effectiveness of terpenes Against Mycoplasma pneumoniae

Terpene beta-ionone or L-carvone was first mixed well with Tween® 80 to have a final Tween® 80 concentration of 5 vol %. This mixture was then used to make concentrations of 2500 ppm in sterile phosphate buffer saline (PBS) by blending the mixture in PBS for 40 seconds. This 2500 ppm solution was then diluted to 500 ppm, 250 ppm, and 125 ppm with PBS.

PBS containing 25 ppm Tween® 80 or PBS alone was used to treat cells suspension as controls.

A log phase (2-3 -day old) culture of Mycoplasma pneumoniae was mixed with each of the above three concentrations of terpene at 1:1 (volume) ratio (in this case, I mL of cell suspension was added to 1 mL of terpene).

The culture and terpene mixture was then incubated at 37° C. for 40 hours. After 40 hours of treatment, 10-fold serial dilution was performed to 10 (−10) by first taking 0.1 mL of the treated culture suspension was added into 0.9 mL of fresh SP4 (Whitcomb (1983); SP4 media is commercially available (Remel, Lenexa, Kans., USA)). All the tubes were then incubated at 37° C., and a color change of the medium was used for the indication of the cells that either were killed or survived from the treatment. Color change was from red to yellow because Mycoplasma pneumoniae produces acid during its growth.

Three days after the 10-fold dilution, the first tube of the following treatments has changed color from red to yellow indication no killing effects:

PBS, PBS containing 25 ppm Tween® 80, 62.5 ppm L-carvone, 125 ppm L-carvone, and 250 ppm L-carvone,

whereas those treated with 62.5 ppm, 125 ppm, and 250 ppm of beta-ionone did not change color at all indicating a killing effect of ionone on Mycoplasma pneumoniae. However, 6 days after the 10-fold dilution, the second and third tube of the PBS, PBS containing 25 ppm Tween® 80, 62.5 ppm L-carvone, 125 ppm L-carvone, and 250 ppm L-carvone changed color,

whereas only the first tube of 62.5 ppm beta-ionone changed color indicating that beta-ionone at 125 and 250 ppm may have completely killed all cells in 40 hours.

All the treatments were performed in duplicate.

### Example 17

Biofilm Formation and Testing (Destruction of Biofilm)

TABLE 12
Terpene formulations.

| Terpene (%) | PL | PL-20 | B | FP | FP-20 | IB | IB-20 |
|---|---|---|---|---|---|---|---|
| Citral | 20 | 20 | 10 | 30 | 30 | 70 | 70 |
| L-carvone | 35 | 35 | 50 | 30 | 30 | 10 | 10 |
| Eugenol | 40 | 40 | 30 | 30 | 30 | — | — |
| B-ionone | — | — | — | — | — | 10 | 10 |
| Tween ® 80 | 5 | — | 10 | 10 | — | 10 | — |
| Span ® 20 | — | 5 | — | — | 10 | — | 10 |

Procedure:

In a 96-well PVC plate

1. Add 100 ml of bacterial culture in nutrient broth, culture has to be made fresh by adding 1-2 ml 1×10⁶ cfu in 50-100 ml broth and incubating overnight (14-18 h) at 37° C.

2. Incubate overnight at 35-37° C. This will develop a Biofilm.

3. Wash 4 times with water.

4. Add 100 ml of 1:1000 terpene solution.

5. Let incubate for 1 hour.

6. Add 25 μl of 1% crystal violet. This is done to quantify the biofilm formation. Dye will coat bacteria attached to wells.

7. Incubate for 15 minutes.

8. Wash wells four times with water and blot dry.

9. Add 200 μl 95% ethanol, mix.

10. In a new plate, transfer 150 μl solution to clean wells.

11. Read at 590 nm.

12. Results are expressed as the difference between O.D. of control as compared to treated samples after subtracting background O.D.

TABLE 13
Results.

| Formula | O.D. Terpene | O.D. Control | % decrease in O.D. |
|---|---|---|---|
| PL | 0.205 | 0.311 | −34 |
| PL-20 | 0.372 | 0.524 | −30 |
| IB | 0.516 | 0.650 | −21 |
| IB-20 | 0.463 | 0.505 | −8 |
| FP | 0.419 | 0.557 | −25 |
| FP-20 | 0.311 | 0.441 | −25 |

### Example 18

Biofilm Formation and Testing (Prevention of Biofilm Formation)

Procedure:

In a 96-well PVC plate

1. Take 50 ml of bacterial culture in nutrient broth, that has made fresh by adding 1-2 ml 1×10⁶ cfu in 50-100 ml broth and incubated overnight (14-18 h) at 37° C.

2. Mix the bacterial broth with 50 μl of 1:1000 terpene solution (as shown in Example 17).

3. Incubate overnight at 35-37° C. This will develop a Biofilm.

4. Wash 4 times with water.

5. Add 50 μl of 1% crystal violet. This is done to quantify the biofilm formation. Dye will coat bacteria attached to wells.

6. Incubate for 15 minutes.

7. Wash wells four times with water and blot dry.

8. Add 200 μl 95% ethanol, mix.

9. In a new plate, transfer 150 μl solution to clean wells.

10. Read at 590 nm.

11. Results are expressed as the difference between O.D. of control as compared to treated samples after subtracting background O.D.

TABLE 14
Results.

| Treatment | O.D. Terpene | O.D. Control | % reduction |
|---|---|---|---|
| IB-20 | 0.059 | 0.278 | 100 |
| FP | 0.044 | 0.266 | 100 |
| B | 0.048 | 0.305 | 100 |
| PL | 0.038 | 0.196 | 98 |
| PL-20 | 0.041 | 0.192 | 96 |
| IB | 0.040 | 0.185 | 98 |

### Example 19

Determination of Best Formula

Study 1:

Procedure: Nutrient agar containing 2.2×10⁸ E. Coli was added to 0.9 ml Butterfield buffer and 1.0 ml of 1:1000 terpene mixture. This mixture was diluted 1:10 four times. The following formulations (from Table 12) were used PL, PL-20, FP, FP-20, IB, IB-20, and control. After mixing (no incubation time), 0.1 ml of the solution was plated on crystal-violet neutral red bile glucose (VRBD) agar and incubated at 37° C. for 18-24 hours.

TABLE 15
Results.

| | | % reduction |
|---|---|---|
| Formula | cfu | from control |
| PL | 5.6 × 10⁵ | 99.50 |
| PL-20 | 1.5 × 10⁶ | 0 |
| FP | 2.9 × 10⁵ | 99.80 |
| FP-20 | 2.4 × 10⁵ | 99.80 |
| IB | 1.0 × 10⁶ | 0 |
| IB-20 | 6.7 × 10⁴ | 99.94 |
| Control | 1.2 × 10⁶ | 0 |

Study 2:

Procedure: Nutrient agar containing 2.2×10⁶ E. coli was added to 0.9 ml Butterfield buffer and 1.0 ml of 1:1000 terpene mixture. This mixture was diluted 1:10 four times. The following formulations were used PL, PL-20, FP, FP-20, IB, IB-20, and control. After mixing (no incubation time) 0.1 ml of the solution was plated on VRBD agar and incubated at 37° C. for 18-24 hours.

TABLE 16
Results.

| | | % reduction |
|---|---|---|
| Formula | cfu | from control |
| PL | 18 | 99.9 |
| PL-20 | 229 | 99.9 |
| FP | 167 | 99.9 |
| FP-20 | 1 | 99.9 |
| IB | 4.5 × 10² | 99.9 |
| IB-20 | 2.3 × 10³ | 99.9 |
| Control | TNC | 0 |

TNC = too numerous to count

Study 3:

Procedure: Nutrient agar containing 2.2×10⁶ E. coli was added to 0.9 ml Butterfield buffer and 1.0 ml of 1:1000 terpene mixture. This mixture was diluted 1:10 twice. The following formulations were used PL, PL-20, FP, FP-20, IB, IB-20, and control. After one hour incubation, 0.1 ml of the solution was plated on VRBD agar and incubated at 37° C. for 18-24 hours.

TABLE 17
Results.

| | | % reduction |
|---|---|---|
| Formula | cfu | from control |
| PL | TNC | 0 |
| PL-20 | TNC | 0 |
| FP | 8.1 × 10³ | 99.9 |
| B | TNC | 0 |
| IB | 6.6 × 10³ | 99.9 |
| IB-20 | TNC | 0 |
| Control | TNC | 0 |

Study 4:

Procedure: Nutrient agar 0.1 ml containing 2.2×10⁶ E. coli was added to 0.9 ml Butterfield buffer and 1.0 ml of 1:1000 terpene mixture. This mixture was diluted 1:10 four times. The following formulations were used PL, PL-20, FP, FP-20, IB, IB-20, and control. After mixing (no incubation time), 0.1 ml of the solution was plated on VRBD agar and incubated at 37° C. for 18-24 hours.

TABLE 18
Results.

| | | % reduction |
|---|---|---|
| Formula | cfu | from control |
| PL | 183 | 99.9 |
| PL-20 | 146 | 99.9 |
| FP | 23 | 99.9 |
| FP-20 | 603 | 99.9 |
| IB | 225 | 99.9 |
| IB-20 | 1.5 × 10⁶ | 50.00 |
| Control | 3.0 × 10⁶ | 0 |

### Example 20

In vitro Effectiveness of Terpenes Against E. coli

This example demonstrates the effect of terpenes on the cell membrane fragility of E. coli, which is considered indicative of other pathogenic bacteria such as Salmonella and Listeria.

Lysis of the cell membrane was monitored by the determination of galactosidase activity. B-galactosidase is a well-characterized cytosolic enzyme in bacteria. This enzyme is inducible in the presence of isopropyl-1-thiogalactosidase (IPTG) and assayed colorimetrically with the substrate o-nitro-phenyl-B-D-galactoside (ONPG). ONPG is cleaved to release o-nitrophenol which has a peak absorbance at 420 nm.

Since intact E. coli is impermeable to both ONPG and the enzyme, the cells have to be lysed prior to enzymatic assay. Therefore, the ability of terpenes to lyse E. coli can be measured with this enzymatic assay and compared to known lysing agents.

The procedure used was as follows: E. coli strains AW574 or AW405 were cultured overnight in 10 ml tryptone broth with 1 nM IPTG at 35° C. Cells were allowed to grow after an absorbance equal to 0.9 was reached.

Cells were harvested, washed with phosphate buffer, and resuspended to an absorbance equal to 0.5.

0.1 ml of the bacteria culture was added to 0.9 ml of buffer, warmed to 30° C., and then 80 μl of terpenes (85 vol % terpenes and 15% polysorbate-80), 80 μl water (background), or 40 μl chloroform plus 40 μl 1% SDS in water (positive control) were added.

After the addition of the lysing agents, the tubes were mixed for 10 seconds, and 0.2 ml of ONPG (4 mg/ml water) was added, then incubated for 5 minutes. The enzyme activity was stopped with 0.5 ml of 1 M sodium carbonate. After being centrifuged for 3 minutes at 1,500×g, the supernatant was transferred to cuvettes and read at 420 nm.

The relative degree of lysis caused by terpenes was calculated as follows:

100×(O.D. terpenes-O.D. water)/(O.D. chloroform−O.D. water).

This shows that dosages can be manipulated to either lyse the cell outright, or in the case of lower dosages, stop bacterial growth without lysis of the cell membrane. The advantage of this controllable result is the ability to prevent lysis and the resultant release of endotoxins where contraindicated.

TABLE 19
Lysis of E. coli by terpenes.

| | Terpenes | Relative lysis |
|---|---|---|
| | (μM) | % |
| Carvone | 404,000 | NM* |
| | 40,400 | 54 |
| | 4,040 | 22 |
| | 404 | 3.2 |
| Geraniol | 363,000 | NM |
| | 36,300 | 96 |
| | 3,630 | 98 |
| | 363 | 34 |
| | 36.3 | 4 |
| | 3.63 | 2.4 |
| b-Ionone | 308,000 | NM |
| | 30,800 | NM |
| | 3,080 | NM |
| | 308 | 52 |
| | 30.8 | 44 |
| | 3.08 | 23 |
| | 0.308 | 4.78 |
| | 0.0308 | 1.3 |

| | |
|---|---|
| 80 μl Polysorbate-80 | 3.2 |
| 80 μl Polysorbate-80 + SDS + Chloroform | 100 |
| SDS + Chloroform | 100* |

*Lysis due to chloroform and SDS combination was considered to be 100%.
*NM = not measurable due to formation of turbid colloidal solution.

### Example 21

In vitro Effectiveness of Terpenes Against Escherichia coli Over Time

This example demonstrates the effectiveness of the terpene mixture, eugenol 40 vol %, L-carvone 35%, citral 20%, and Tween® 80 5%, at several concentrations against Escherichia coli and cultured over time.

Terpene dilutions (1:500, 1:1000, 1:2000, 1:4000, 1:8000, and 1:16,000) were prepared in brain heart infusion (BHI) broth and in saline. These were prepared in 25 ml amounts.

E. coli was grown overnight in BHI broth and diluted to a MacFarland 0.5 concentration in saline. This solution was diluted 1:100 to be used to inoculate (0.5 ml) each terpene dilution tube.

The series that contained the terpene dilution in BHI was tested at 30 min., 90 min., 150 min., and 450 min. Each tube was mixed and serially diluted in saline. 0.5 milliliters of each dilution was spread plated onto MacConkey (MAC) agar plates. Also, 3 drops of the undiluted and the 1:100 dilution was added into respective tubes of BHI broth. The tubes and plates were incubated overnight at 35° C.

The series that contained the terpene dilution in saline was tested at 60 min., 120 min., 180 min., and 480 min. Each tube was mixed and serially diluted in saline. 0.5 milliliters of each dilution was spread plated onto MacConkey (MAC) agar plates. Also, 3 drops of the undiluted and the 1:100 dilution were added into respective tubes of BHI broth. The tubes and plates were incubated overnight at 35° C.

TABLE 20
Subculture from the tubes containing various dilutions of
terpenes in broth.

| Time | Dilution | 1:500 | 1:1000 | 1:2000 | 1:4000 | 1:8000 | 1:16,000 |
|---|---|---|---|---|---|---|---|
| 30 | Un- | NG | + | + | + | + | + |
| min | diluted | | | | | | |
| | 1:100 | NG | + | + | + | + | + |
| 90 | Un- | NG | NG | + | + | + | + |
| min | diluted | | | | | | |
| | 1:100 | NG | NG | NG | + | + | + |
| 150 | Un- | NG | NG | + | + | + | + |
| min | diluted | | | | | | |
| | 1:100 | NG | NG | NG | + | + | |
| 450 | Un- | NG | NG | + | + | + | + |
| min | diluted | | | | | | |
| | 1:100 | NG | NG | + | + | + | + |

NG = no growth, + = growth

TABLE 21
Subculture from the tubes containing various dilutions of
terpenes in saline.

| Time | Dilution | 1:500 | 1:1000 | 1:2000 | 1:4000 | 1:8000 | Control |
|---|---|---|---|---|---|---|---|
| 60 | Undiluted | NG | + | + | + | + | + |
| min | 1:100 | NG | NG | NG | + | + | + |
| 120 | Undiluted | NG | NG | NG | + | + | + |
| min | 1:100 | NG | NG | NG | NG | + | + |
| 180 | Undiluted | NG | NG | NG | + | + | + |
| min | 1:100 | NG | NG | NG | NG | + | + |
| 480 | Undiluted | NG | NG | NG | NG | + | + |
| min | 1:100 | NG | NG | NG | NG | + | + |

NG = no growth, += growth

TABLE 22
The quantitative results of the activity of various terpene dilutions
against E. coli (cfu).

| Media | Time | 1:500 | 1:1000 | 1:2000 | 1:4000 | 1:8000 | Control |
|---|---|---|---|---|---|---|---|
| Broth | 30 | 0 | 0 | 660 | 3600 | 3600 | 4600 |
| | min | | | | | | |
| | 90 | 0 | 0 | 12 | 4600 | 5400 | 7600 |
| | min | | | | | | |
| | 150 | 0 | 0 | 10 | 8000 | 12,000 | 14,000 |
| | min | | | | | | |
| | 450 | 0 | 0 | 15,000 | 28 × | 23 × | 16 × |
| | min | | | | 10³ | 10⁷ | 10⁸ |
| Saline | 60 | 0 | 4 | 140 | 4000 | 2000 | 1300 |
| | min | | | | | | |
| | 120 | 0 | 0 | 0 | 90 | 3800 | 2600 |
| | min | | | | | | |
| | 180 | 0 | 0 | 0 | 2 | 2000 | 5000 |
| | min | | | | | | |
| | 480 | 0 | 0 | 0 | 0 | 104 | 8000 |
| | min | | | | | | |

NG = no growth, + = growth

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of decreasing pathogen and/or parasite concentration in a room or on a surface comprising applying a composition comprising an effective amount of at least one effective terpene.

2. A method of improving air quality in a confined space comprising applying a composition comprising an effective amount of at least one effective terpene.

3. The method of claim 1 wherein the composition is a solution.

4. The method of claim 1 wherein the composition further comprises water.

5. The method of claim 1 wherein the composition further comprises a surfactant and water.

6. The method of claim 5 wherein the surfactant is polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, Span® 20, Span® 40, Span® 60, Span® 80, or mixtures thereof.

7. The method of claim 1 wherein the composition further comprises saline or a buffer solution.

8. The method of claim 1 wherein the at least one terpene is a mixture of different terpenes.

9. The method of claim 1 wherein the at least one terpene is a terpene-liposome combination.

10. The method of claim 1 wherein the terpene comprises citral, pinene, nerol, b-ionone, geraniol, carvacrol, eugenol, carvone, terpeniol, anethole, camphor, menthol, limonene, nerolidol, famesol, phytol, carotene (vitamin A₁), squalene, thymol, tocotrienol, perillyl alcohol, borneol, myrcene, simene, carene, terpenene, linalool, or mixtures thereof.

11. The method of claim 1 wherein the terpene is citral, carvone, b-ionone, eugenol, eucalyptus oil, or mixtures thereof.

12. The method of claim 1 wherein composition comprises about 1 to 99% by volume terpenes and about 1 to 99% by volume surfactant.

13. The method of claim 1 wherein the terpene comprises between about 100 ppm and about 2000 ppm.

14. The method of claim 1 wherein the terpene comprises about 100 ppm.

15. The method of claim 1 wherein the terpene comprises about 250 ppm.

16. The method of claim 1 wherein the terpene comprises about 500 ppm.

17. The method of claim 1 wherein the terpene comprises about 1000 ppm.

18. The method of claim 1 wherein the terpene is 50% L-carvone, 30% eugenol, 10% purified eucalyptus oil and the effective amount is 1000 ppm, and wherein 10% is a surfactant.

19. The method of claim 1 wherein the terpene is citral and the effective amount is 1000 ppm.

20. The method of claim 19 wherein the composition further comprises 5% surfactant.

21. The method of claim 1 wherein the terpene is b-ionone and the effective amount is 250 ppm and wherein 5% is a surfactant.

22. The method of claim 1 wherein the terpene is effective against bacteria, mycoplasmas, fungi, and/or parasites.

23. The method of claim 1 wherein the terpene is effective against bacteria.

24. The method of claim 1 wherein the terpene is effective against mycoplasmas.

25. The method of claim 1 wherein the terpene is effective against parasites.

26. The method of claim 5 wherein the terpene is 10 vol % b-ionone, 10% L-carvone, and 70% citral and wherein the surfactant is 10 vol %.

27. The method of claim 5 wherein the terpene is 20 vol % citral, 35% L-carvone, and 40% eugenol and wherein the surfactant is 5 vol %.

28. The method of claim 5 wherein the terpene is 70 vol % citral, 10% L-carvone, and 10% eugenol and wherein the surfactant is 10 vol %.

29. The method of claim 25 wherein the terpene is about 50 μM b-ionone.

30. The method of claim 1 wherein the composition is a pressurized or foaming composition.

31. The method of claim 1 wherein the composition is an aerosol.

32. The method of claim 1 wherein the application is by spraying.

33. The method of claim 1 wherein the composition is a true solution.

34. A method of improving air quality in a confined space by decreasing pathogen and/or parasitic concentration comprising applying a composition comprising an effective amount of an effective terpene.

35. The method of claim 34 wherein the composition is a pressurized or foaming solution.

36. The method of claim 34 wherein the composition further comprises a surfactant.

37. The method of claim 34 wherein the confined space is a closed room and/or its surfaces.

38. The method of claim 36 wherein the composition comprises about 1 to 100 vol % terpenes and about 0 to 99% surfactant.

39. The method of claim 34 wherein the terpene comprises citral, b-ionone, geraniol, carvacrol, eugenol, carvone, terpeniol, anethole, or combinations thereof.

40. The method of claim 36 wherein the surfactant comprises non-ionic or anionic surfactant.

41. The method of claim 36 wherein the surfactant comprises polysorbate-80, polysorbate-20, polysorbate-40, polysorbate-60, polyglyceryl ester, polyglyceryl monooleate, decaglyceryl monocaprylate, propylene glycol dicaprilate, triglycerol monostearate, or a combination thereof.

42. The method of claim 34 wherein the pathogen or parasite is Sclerotinta sp., Rhizoctonia sp., Colletotrichum sp., Mucor sp., Paecilomyces sp., or combinations thereof.

43. A method of improving air quality by decreasing pathogen and parasite concentrations in closed rooms and surfaces comprising applying a pressurized or foaming solution comprising an effective amount of an effective terpene, an effective terpene mixture, a liposome- effective terpene(s) composition, or combination thereof.

44. A method for preventing a respiratory infection comprising
decreasing pathogen and/or parasite concentration in a room or on a surface using the method of claim 1.

45. A method for preventing a respiratory infection comprising
improving air quality in a confined space containing a subject using the method of claim 2.

46. The method of claim 45 wherein the subject is livestock.

47. The method of claim 45 wherein the subject is human.
